(19) United States
(12) Patent Application Publication
(10) Pub. No.: US 2023/0193367 A1
(43) Pub. Date: Jun. 22, 2023

(54) METHOD FOR MULTIPLYING DNA, ROTATION DEVICE AND SYSTEM FOR MULTIPLYING DNA

(71) Applicant: SpinDiag GmbH, Freiburg (DE)

(72) Inventors: Frank Schwemmer, Freiburg (DE); Gregor Gross-Czilwik, Freiburg (DE)

(21) Appl. No.: 18/171,736

(22) Filed: Feb. 21, 2023

Related U.S. Application Data
(63) Continuation of application No. PCT/EP2020/073202, filed on Aug. 19, 2020.

(51) Int. Cl. C12Q 1/6844; B01L 7/00; B01L 3/00

(52) U.S. Cl. CPC C12Q 1/6844; B01L 3/50851; B01L 7/52; B01L 7/54; B01L 2300/0609; B01L 2300/1822; B01L 2400/0409; B01L 2400/0445

(57) ABSTRACT

A method for multiplying DNA includes using a rotation device to rotate a sample carrier about an axis of rotation. The sample carrier has at least one cavity in which a sample liquid containing DNA is received. The cavity is heated to a high temperature value only on a heat input side lying in a rotation plane by using a heating device. As a result of the heating, a convection current is created in the sample liquid in the cavity, the convection current having substantial current components directed perpendicularly to the rotation plane. A circulation time of a liquid particle along a current path of the convection current is predetermined by the speed of the rotation. A rotation device for multiplying DNA and a system for multiplying DNA, are also provided.

### CROSS-REFERENCE TO RELATED APPLICATION

This application is a continuation, under 35 U.S.C. § 120, of copending International Patent Application PCT/EP2020/073202, filed Aug. 19, 2020, which designated the United States; the prior application is herewith incorporated by reference in its entirety.

### FIELD AND BACKGROUND OF THE INVENTION

The invention relates to a method for multiplying DNA and to a rotation device which is preferably configured and intended for carrying out the method. The invention further relates to a system for multiplying DNA.

Besides scientific genetic material analyses, paternity tests and the like, DNA (deoxyribonucleic acid) is commonly analyzed to test for diseases present or detected, to detect pathogens. Starting from a sample - for example a swab, a blood sample or the like - that requires multiplication of specific regions of a DNA (optionally also RNA) contained therein. When detecting or analyzing RNA in a sample (e.g., for detection of a virus), it is first transcribed into DNA by so-called “reverse transcription” and then multiplied.

In order to multiply the DNA, use is usually made of the so-called polymerase chain reaction (PCR for short) in a liquid reaction. The DNA is typically present in the form of a double-helix structure formed of two complementary single strands of DNA. In the PCR, the DNA is first separated into two single strands by an increased temperature of the liquid reaction between typically 90-96° C. (“denaturation phase”).

Thereafter, the temperature is lowered again (“annealing phase,” typically within a range of 50-70° C.) in order to allow specific annealing of so-called primer molecules on the single strands. The primer molecules are short complementary DNA strands which attach to a defined site on the single strands of DNA. The primers serve as the starting point for an enzyme, the so-called polymerase, which, in the so-called elongation phase, fills in the building blocks (dNTPs) complementary to the available DNA sequence of the single strand. Starting from the primer molecule, that gives rise to a doublestranded DNA again. Elongation is typically carried out at the same temperature as in the annealing phase or at a slightly elevated temperature, typically between 65 and 75° C. Following elongation, the temperature is increased again for the denaturation phase.

That cycling of the temperature in the liquid reaction between the two to three temperature ranges is called PCR thermocycling and is typically repeated in 30 and 50 cycles. In every cycle, the specific region of DNA is multiplied. Typically, the thermocycling of the liquid reaction in a reaction vessel is realized by controlling the external temperature. The reaction vessel is, for example, present in a thermal block in which the PCR thermocycling is realized by heating and cooling of a solid body in thermal contact with the reaction vessel, and in so doing supply and dissipate heat from the liquid. Alternative heating and cooling concepts for realizing PCR thermocycling are, inter alia, controlling the temperature of fluids (especially air and water) that flow around the reaction vessel, and radiation-based concepts, for example by introducing heat by UV radiation or laser radiation.

In the case of a customary polymerase chain reaction, the process times are within the range of several minutes and are therefore comparatively time-consuming.

### SUMMARY OF THE INVENTION

It is accordingly an object of the invention to provide a method for multiplying DNA, a rotation device and a system for multiplying DNA, which overcome the hereinafore-mentioned disadvantages of the heretofore-known methods, devices and systems of this general type and which quicken a polymerase chain reaction.

This object is achieved according to the invention by a method for multiplying DNA, a rotation device and a system. Further embodiments and developments of the invention that are advantageous and are in some cases in themselves inventive are stated in the dependent claims and in the following description.

With the foregoing and other objects in view there is provided, in accordance with the invention, a method for multiplying DNA. According to the method, a sample carrier having at least one cavity for accommodating a sample liquid is preferably first filled with a sample liquid containing DNA in such a way that the sample liquid is accommodated in the cavity. Thereafter, the sample carrier is rotated about a rotation axis by using a rotation device. The cavity, preferably the sample carrier, is heated by using a heating device to a high temperature value only on a heat input side lying in a rotation plane (i.e., especially parallel to a rotation plane). Preferably, the side opposite the heat input side is not heated. The heating generates a convection current of the sample liquid inside the cavity. The convection current has substantial current components directed (at least mainly) perpendicularly to the rotation plane, i.e., from the heat input side to the opposite side referred to hereinafter as “heat output side,” of the sample carrier and/or reversely. Preferably, the convection current is generated substantially annularly, with a first current segment especially extending approximately parallel to the heat input side, a second current segment from the heat input side to the heat output side, a third current segment parallel to the heat output side, and a fourth current segment back to the heat input side again (from the heat output side). As a result, the sample liquid is preferably guided through a denaturation zone (having especially a high temperature value), a so-called annealing zone (or: primer hybridization zone) and an extension zone, and back to the denaturation zone. In addition, a circulation time of a liquid particle of the sample liquid along a current path of the convection current is specified (in particular “controlled”) by the speed of rotation.

In particular, the circulation time of the liquid particle is additionally also influenced by further parameters, for example the geometry of the cavity, the viscosity of the sample liquid, the density of the sample liquid, the ensuing temperature gradient, and the like. In this case, the speed is, however, a parameter that is alterable comparatively easily and quickly (and in view of geometry in any case).

In other words, due to the above-described one-sided heating of the cavity, a temperature gradient (which is therefore oriented in decreasing direction from the heat input side to the heat output side) is preferably formed perpendicularly to a dominating force, in particular the centrifugal force resulting from the rotation, on the sample liquid in the cavity.

In this case and hereinafter, “substantial current components” is especially understood to mean that the current components have a non-negligible share of the volume of the sample liquid flowing in the convection current. This means that the current components are not just partial currents that occur randomly and that occur locally and possibly for a limited time. For example, the share of such a perpendicular current component is up to about a quarter of the total flowing volume. In particular, what occurs is a fluid exchange, necessary for the polymerase chain reaction, between the denaturation zone and the annealing zone through these current components or current segments that are directed mainly perpendicularly to the rotation plane. “Mainly perpendicularly” is especially understood to mean that the current segments are exactly or at least approximately (e.g., with an inclination of up to 30 degrees) perpendicular to the rotation plane.

Preferably, what are additionally also present however, besides the above-described four current segments, are components that flow transversely thereto due to the centrifugal force and/or the Coriolis force. This advantageously leads to additional mixing of the sample liquid, and so it is possible for reaction partners - i.e., DNA to be multiplied, primer molecules and “strand building blocks” - to be mixed as homogenously as possible.

In this case and hereinafter, the term “circulation time” is especially understood to mean the time required by the (especially infinitesimal) liquid particles to flow through the denaturation zone, the annealing zone (or: primer hybridization zone) and the extension zone back to the denaturation zone. The circulation time can be set to times in the range between 0.1 s and 20 s by the speed (i.e., by the rotational speed). Inside the cavity - corresponding to a reaction chamber of the sample carrier - it is thus possible set an average current speed in the order of magnitude of up to 22 mm/s.

Such a short circulation time and/or such a high current speed makes a particularly rapid polymerase chain reaction possible, and so it is advantageously possible to save process time.

In a preferred method variant, the cavity is cooled on the heat output side opposite the heat input side to a low temperature value compared to the high temperature value on the heat input side. As a result, it is advantageously possible to set the temperature of the annealing zone (and the extension zone) and especially prevent the sample liquid in the region of the annealing zone from increasingly heating.

In a preferred method variant, heating is achieved by applying a constant temperature value to the heat input side by using the heating device. Optionally, cooling is analogously likewise achieved by applying a constant temperature value to the heat output side. This dispenses with (usually cyclic) heating and cooling phases which, in the case of conventional polymerase chain reactions, lead to a comparatively long (total) time for multiplying DNA. In addition, the procedure for the polymerase chain reaction is simplified, since “ramp functions” are not necessary, it being necessary instead to only regulate to one target value (high or low temperature value). Likewise, the structure of the heating device and possibly also the rotation device can be kept simple.

Preferably, the specified temperature value of the heating device is a value between 80 and 110° C., in particular between 90 and 100° C., and so what is established in the denaturation zone is a temperature value above the DNA melting temperature. On the heat output side, a temperature value of in particular about 10 to 60, preferably 40° C. is applied, and so what is established in the annealing or extension zone (which are preferably disposed within the same region on the heat output side) is a temperature value of from 50 to 70, in particular 60° C.

Preferably, cooling is achieved by utilizing a stream of cooling air. It can be generated by using comparatively simple measures, for example some kind of processor fan, a (for example cooling) fan or the like.

Further preferably, the heating is performed by using the heating device spanning at least the base of the cavity disposed on the heat input side. This means that the heating device used preferably has through a planar heating element. The planar extent of the heating device preferably extends over a surface area larger than the base of the cavity, preferably over a surface area many times larger. As a result, it is advantageously possible to heat multiple cavities (of the same sample carrier or else multiple sample carriers) at the same time and thus increase the throughput. Preferably, the heating device is integrated into a sample holder of the rotation device that supports the sample carrier.

In a convenient method variant, the convection current is guided inside the cavity by a flow resistance assigned to the cavity. As a result, it is possible to locally alter the flow speed and/or the pressure.

In a preferred method variant, the convection current is guided by using the above-described flow resistance in such a way that one part of the current path that is directed from the heat input side to the heat output side runs especially only on a side of the cavity that is nearest the rotation axis and the part of the current path that is directed from the heat output side to the heat input side runs especially only on the side of the cavity that is remote from the rotation axis. Preferably, the flow resistance is chosen and adjusted in such a way that the sample liquid in the regions between the heat input side and the heat output side, as opposed to the (warmer and colder) regions assigned to the heat input side and the heat output side (i.e., in particular the denaturation zone and the annealing zone and extension zone), is counteracted by an at least doubled current resistance. Optionally, the flow resistance is also chosen and adjusted in such a way that a larger partial volume of the cavity is assigned to the colder region, and so the sample liquid can reside for longer in the region than in the warmer region. As a result of this control, it is thus advantageously possible to specify the residence time of the liquid particles in the respective region, i.e., preferably the extension time.

In a preferred embodiment, the cavity has an approximately cuboid geometry. The flow resistance is preferably formed by some kind of beam or transverse web and divides the cavity especially into at least one flow channel from the heat input side to the heat output side on a radial inner side of the cavity and at least one flow channel from the heat input side to the heat output side on a radial outer side of the cavity. Fluidically coupled to one another by these two flow channels are the warmer and colder partial volumes of the cavity (that are respectively assigned to the heat input side and to the heat output side). Optionally, each of the two flow channels is divided once more into subchannels with the aid of webs.

In a further convenient method variant, the structure of the sample carrier in the vicinity of the cavity is appropriately chosen to influence (control) the convection current. In order to influence the heat input by the heating device and the heat output on the heat output side (optionally toward the cooling device) and especially to specify the resultant thermal conductivity, what are especially appropriately chosen are the geometry, the wall thickness and/or the material of the sample carrier. A comparatively thick wall composed of plastic, for example polycarbonate or polymethyl methacrylate, leads to a comparatively low thermal conductivity. The addition of thermally conductive fillers (carbon black, ceramic or the like) increases thermal conductivity at the same wall thickness.

In a further preferred method variant, a sample carrier having a plurality of cavities for parallel multiplication of DNA is used. As a result, it is advantageously possible to increase the throughput and thus the quantity of the multiplied DNA. Additionally or alternatively, it is also possible to already assign different primers and/or probes to various cavities in a “dry” state (i.e., prior to filling with sample liquid). This allows parallel detection of various target DNA segments in respectively assigned cavities.

Optionally, the above-described method is used in the context of a multistage multiplication for a first multiplication stage (“preliminary stage”) and/or a second multiplication stage (main multiplication). Optionally, the sample carrier also has different cavities for the respective stage, and so the samples assigned to the respective stage can be multiplied at the same time (with subsequent “movement” into the cavity of the next-highest stage).

With the objects of the invention in view, there is also provided a rotation device configured and intended for use in multiplying DNA, especially in the context of the above-described method. To this end, the rotation device includes a process chamber and a sample holder disposed in the process chamber. The sample holder is configured and intended for holding at least one sample carrier of the kind described above. The sample carrier therefore has at least one cavity (of the kind described above) which is used for accommodating the DNA-containing sample liquid. Furthermore, the rotation device includes a rotational drive through the use of which the sample holder is rotated about the (aforementioned) rotation axis during intended operation. In addition, the rotation device includes the aforementioned heating device through the use of which the heat input side of the sample carrier, of at least the cavity, lying in the rotation plane of the sample holder is heated to a high temperature value during intended operation. Furthermore, the rotation device includes a controller which is control-linked to the rotational drive and the heating device and configured to carry out the above-described method for multiplying DNA especially automatically, optionally in cooperation with laboratory personnel.

The rotation device and the above-described method share the above-described advantages and especially also the physical features possibly described in the context of the method.

In the context of the invention, the controller (optionally also referred to as “control unit”) can be in the form of a nonprogrammable electronic circuit. Preferably, the controller is, however, formed by a microcontroller in which the functionality for carrying out the method according to the invention is implemented in the form of a software module. Optionally, the microcontroller and/or the software module is realized in the context of a separate control computer.

Preferably, the sample holder is some kind of plate (or: disk or round plate) on which the sample carrier for carrying out the method can be fastened. For fastening, the sample holder optionally includes a clamping device, for example clamps, some kind of clamping jaw or the like.

In a convenient embodiment, the heating device includes Peltier elements. Alternatively, the heating device includes a resistance heating element, ceramic heating or the like. Radiation-based heating - for example an infrared heater - is optionally used, too. Preferably, the heating device is extended planarly, and so it can especially cover multiple cavities of one or more sample carriers.

Particularly preferably, the heating device is integrated into the sample holder; it is at least set therein - for example inserted into an appropriately dimensioned recess of the sample holder. This allows a compact construction.

In a convenient embodiment, the rotation device includes the above-described cooling device for cooling the cavity on the heat output side opposite the heat input side to a low temperature value.

In a convenient variant, the cooling device is formed by the (cooling) fan. Through the use of the fan, cooling air preferably flows through the process chamber during intended use. In this case, the fan is preferably also used for cooling the rotational drive. Optionally, the fan is disposed in the process chamber in such a way that flow is received by the heat output side of the sample carrier. This may be advantageous if movement of air away from the sample carrier, caused by centrifugal force due to the rotation of the sample carrier, is insufficient for cooling. Alternatively, the cooling device can, however, also be formed by a cooling plate which is placed onto the sample carrier on the heat output side thereof. The cooling plate preferably includes Peltier elements which are used for cooling.

Optionally, the above-described fan also has a cooling function, for example in the manner of a refrigerator, an air-conditioning system or the like. In this case, the rotation device can advantageously also be operated in a comparatively warm environment. Alternatively, “only” ambient air is blown into the process chamber by using the fan. In this case, the temperature of the process chamber is optionally constantly adjusted by regulation of the fan speed by using a temperature sensor.

In this case and hereinafter, the heat input side is especially understood to mean one side, preferably the bottom side of the sample carrier and thus also of the respective cavity. During intended operation, the bottom side rests on the sample holder and thus on the heating device. Accordingly, the heat output side refers especially to the top side of the sample carrier. In addition, the terms heat input side and heat output side can also be assigned to the corresponding sides of a partial volume inside the process chamber that is intended for the sample carrier.

In a further convenient embodiment, the rotation device also includes a fluorescence detector for detecting sufficient multiplication of the DNA. To this end, there is preferably added to the sample liquid an (especially initially inactive) dye, the fluorescence of which, for example, increases with increasing number of multiplied DNA strands (and thus decreasing number of free reaction partners). Therefore, the fluorescence inside the cavity is a measure of the conversion achieved.

With the objects of the invention in view, there is concomitantly provided a system for multiplying DNA. The system comprises the above-described rotation device and the at least one above-described sample carrier.

In this case and hereinafter, the conjunction “and/or” is especially to be understood to mean that the features linked by the conjunction can be formed either jointly or as alternatives to one another.

Other features which are considered as characteristic for the invention are set forth in the appended claims.

Although the invention is illustrated and described herein as embodied in a method for multiplying DNA, a rotation device and a system for multiplying DNA, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 is a diagrammatic, side-elevational view of a system for multiplying DNA, including a rotation device and a sample carrier;

FIG. 2 is a sectional view of a detail of the sample carrier and a sample holder of the rotation device;

FIG. 3 is a view similar to FIG. 2 of an alternative exemplary embodiment of the sample carrier;

FIG. 4 is a top-plan view of the sample carrier of FIG. 3;

FIG. 5 is a view similar to FIG. 4 of a further exemplary embodiment of the sample carrier; and

FIG. 6 is a flow chart of a method for multiplying DNA.

### DETAILED DESCRIPTION OF THE INVENTION

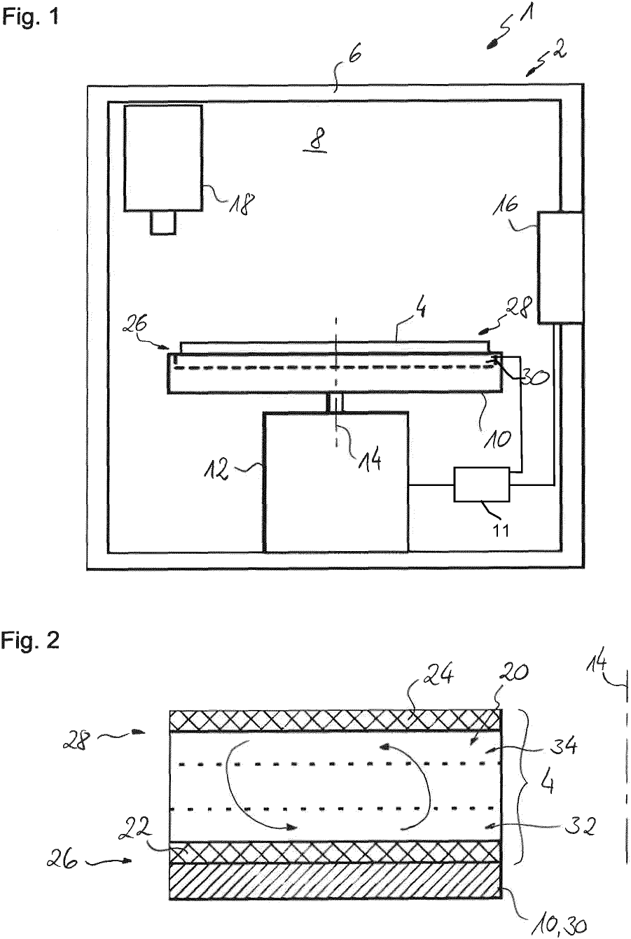

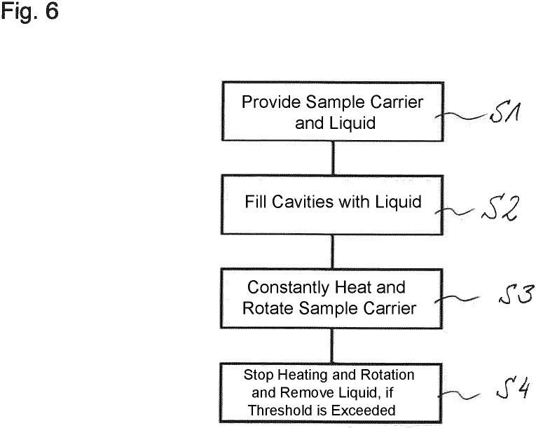

Referring now in detail to the figures of the drawings, in which parts corresponding to one another are always provided with the same reference signs, and first, particularly, to FIG. 1 thereof, there is seen a system 1 for multiplying DNA. The system 1 includes a rotation device 2 and a sample carrier 4. What is carried out by using the system 1 is a method for multiplying DNA that will be described in greater detail below on the basis of FIG. 6.

The rotation device 2 includes a housing 6 which surrounds a housing interior, referred to hereinafter as “process chamber 8.” Furthermore, the rotation device 2 includes a sample holder 10. Mounted thereon is the sample carrier 4 when the method is carried out (i.e., during intended operation). The sample holder 10 is rotatable about a rotation axis 14 by using a rotational drive 12. The sample holder 10 is therefore a rotary plate. Furthermore, the rotation device 2 includes a fan 16 as a cooling device, through the use of which a stream of cooling air flows through the process chamber 8 during intended operation. In addition, the rotation device 2 includes a fluorescence detector 18.

The sample carrier 4 has at least one cavity 20 (see FIG. 2) for accommodating a DNA-containing sample liquid. In a preferred exemplary embodiment, the sample carrier 4 has two or more of the cavities 20. The cavity 20 has a cuboid shape having, for example, dimensions of about 5×3×1.2 mm³ and is delimited by a bottom wall 22 and a top wall 24 in relation to the bottom side (hereinafter: “heat input side 26”) and the top side (hereinafter: “heat output side 28”), respectively, and by lateral walls in relation to the remaining sides, which lateral wall are not depicted in greater detail. The walls of the sample carrier 4 are made of plastic, specifically a cyclic olefin copolymer (COC). During intended operation, the heat input side 26 of the sample carrier 4 is positioned on the sample holder 10.

The rotation device 2 includes a heating device 30. It in turn includes a Peltier element which extends planarly over the top side of the sample holder 10 that faces the heat input side 26, optionally multiple Peltier elements positioned side-by-side for planar heat emission. The heating device 30 is integrated into the sample holder 10. In an exemplary embodiment not depicted in greater detail, an aluminum plate for homogenous temperature distribution is disposed between the Peltier element and the sample holder 10.

A controller 11 of the rotation device 2 for controlling the rotational drive 12, the heating device 30 and the fan 16 is present.

In order to multiply DNA, the sample carrier 4 and the DNA-containing sample liquid is provided in a first method step S1 (see FIG. 6). The sample liquid contains not only the DNA to be multiplied, but also primer molecules, structural building blocks for forming new DNA strands, and polymerase. In a second method step S2, the cavities 20 are filled with the sample liquid.

In a third method step S3, the sample carrier 4 is kept constant at a high temperature value of about 95° C. on the heat input side 26 by using the heating device 30. In parallel, the rotational drive 12 drives the sample holder 10 for rotation about the rotation axis 14, and so each cavity 20 is rotated about the rotation axis 14. By using the fan 16, a stream of cooling air (of preferably 40° C.) is blown over the sample carrier 4, and so the heat output side 28 thereof is kept constant at this low temperature value.

Due to the bottom-sided heating and the top-sided cooling, what are formed inside the cavity 20 are a warm region 32 and a cold region 34 (indicated by dotted lines), i.e., a temperature gradient which runs parallel to the rotation axis 14. In the cold region 34, the sample liquid has a temperature value of about 60° C. In the warm region 32, the temperature value of the sample liquid lies above the melting temperature of the DNA, specifically above 90° C.

Due to the bottom-sided heating and the top-sided cooling, what is established is a buoyancy-driven convection current, based on the temperature-related density differences of the sample liquid. The convection current is basically annular (namely approximately in the form of an oval, cf. semicircular arrows in FIG. 2) and oriented with current components approximately perpendicular to the rotation plane of the sample holder 10. Due to the centrifugal forces of the rotation (directed to the right in FIG. 2) and to the Coriolis force likewise present due to the rotation, what however also occurs is (homogeneous) mixing of the sample liquid transversely to the basic current path of the convection current. The speed of the convection current increases as the speed of rotation increases.

In the course of the convection current, the sample liquid thus passes (approximately parallel to the rotation plane) through the warm region 32, in which the temperature causes denaturation of the DNA. Therefore, the warm region 32 is also referred to as the “denaturation zone.” After flowing to the heat output side 28 in a direction approximately perpendicular to the rotation plane, the sample liquid passes (again approximately parallel to the rotation plane) through the cold region 34, in which primer hybridization and then extension of the DNA strands take place. The cold region 34 is therefore also referred to as the annealing or extension zone. After passage through the cold region 34, the sample liquid flows (approximately perpendicularly to the rotation plane) back to the warm region 32.

The method step S3 is maintained until the fluorescence detector 18 determines a sufficiently high conversion of the structural building blocks, etc., intended for the multiplication. To this end, what is specifically carried out is a threshold comparison between a value of the measured fluorescence and a threshold specified (e.g., empirically determined) for a sufficiently high conversion. If the threshold is exceeded, the rotation of the sample holder 10 and the heating by using the heating device 30 are stopped, and the sample liquid is removed from the respective cavity 20, in a fourth method step S4.

Alternatively, the method step S3 is terminated after a specified time. The time plot of the fluorescence is optionally used to estimate the concentration of the DNA in the original sample.

The method steps S1 to S3 in particular can also be at least partially simultaneously carried out. In particular, the sample holder 10 need not stand still during the filling of the cavities 20. Similarly, the heating device 30 can also be already heating the heat input side 26.

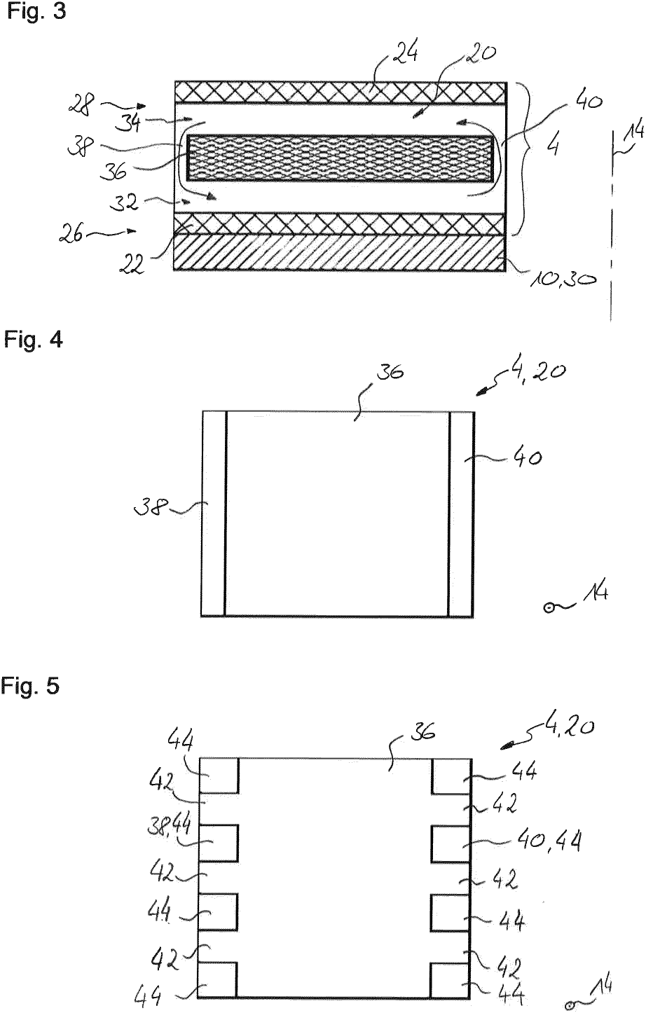

FIGS. 3 and 4 depict an alternative exemplary embodiment of the sample carrier 4 having a modified structure of the respective cavity 20. Disposed inside the cavity 20 is a flow resistance 36 in the form of a beam or cuboid extending parallel to the rotation plane through the cavity 20. The flow resistance 36 is disposed in such a way that a radially inner (directed to the rotation axis 14) first flow channel 38 and a radially outer flow channel 40 are kept clear, and the current path of the convection current runs through the channels. Therefore, the flow resistance 36 separates the warm region 32 from the cold region 34, with the exception of the flow channels 38 and 40.

In the exemplary embodiment depicted, the flow channels 38 and 40 have the same channel cross section. In addition, the warm and cold regions 32 and 34 have the same dimensions.

In an optional exemplary embodiment (not depicted in greater detail), the flow resistance 36 is disposed in such a way that a larger partial volume of the cavity 20 is assigned to the cold region 34 than to the warm region 32. This achieves a higher extension time (residence time in the cold region 34, i.e., the extension zone).

Further optionally, the flow channels 38 and 40 have different channel cross sections.

FIG. 5 depicts a further exemplary embodiment of the cavity 20. The flow resistance 36 divides the respective flow channels 38 and 40 into subchannels 44 by using further webs 42. The subchannels 44 respectively assigned to the flow channels 38 and 40 can again have different cross sections.

The subject matter of the invention is not restricted to the above-described exemplary embodiments. Rather, further embodiments of the invention can be derived from the above description by a person skilled in the art. In particular, the individual features of the invention described on the basis of the various exemplary embodiments and their structural variants can also be combined with one another in another way.

The following is a summary list of reference numerals and the corresponding structure used in the above description of the invention.

List of Reference Signs:

| | |
|---|---|
| 1 | System |
| 2 | Rotation device |
| 4 | Sample carrier |
| 6 | Housing |
| 8 | Process chamber |
| 10 | Sample holder |
| 12 | Rotational drive |
| 14 | Rotation axis |
| 16 | Fan |
| 18 | Fluorescence detector |
| 20 | Cavity |
| 22 | Bottom wall |
| 24 | Top wall |
| 26 | Heat input side |
| 28 | Heat output side |
| 30 | Heating device |
| 32 | Region |
| 34 | Region |
| 36 | Flow resistance |
| 38 | Flow channel |
| 40 | Flow channel |
| 42 | Web |
| 44 | Subchannel |
| S1 | Method step |
| S2 | Method step |
| S3 | Method step |
| S4 | Method step |
| S5 | Method step |

1. A method for multiplying DNA, the method comprising:
using a rotation device to rotate a sample carrier about a rotation axis, the sample carrier having at least one cavity in which a sample liquid containing DNA has been accommodated;
using a heating device to heat the cavity to a high temperature value only on a heat input side of the cavity lying in a rotation plane;
carrying out the heating to generate a convection current of the sample liquid inside the cavity, the convection current having substantial current components directed perpendicularly to the rotation plane; and
using a speed of rotation of the sample carrier to specify a circulation time of a liquid particle along a current path of the convection current.

2. The method according to claim 1, which further comprises cooling the cavity on a heat output side of the cavity to a temperature value lower than the temperature value on the heat input side, the heat output side being opposite the heat input side.

3. The method according to claim 2, which further comprises at least one of carrying out the heating by applying a constant temperature value to the heat input side or carrying out the cooling by applying a constant temperature value to the heat output side.

4. The method according to claim 2, which further comprises carrying out the cooling by applying a stream of cooling air.

5. The method according to claim 1, which further comprises carrying out the heating with the heating device spanning at least a base of the cavity disposed on the heat input side.

6. The method according to claim 5, which further comprises integrating the heating device into a sample holder of the rotation device supporting the sample carrier.

7. The method according to claim 1, which further comprises guiding the convection current inside the cavity by a flow resistance assigned to the cavity.

8. The method according to claim 7, which further comprises carrying out the guiding of the convection current by the flow resistance in such a way that:
a part of a current path directed from the heat input side to the heat output side runs on a side of the cavity nearest to the rotation axis, and
a part of the current path directed from the heat output side to the heat input side runs on a side of the cavity remote from the rotation axis.

9. The method according to claim 1, which further comprises providing the sample carrier with a plurality of cavities for parallel multiplication of DNA.

10. A rotation device for multiplying DNA, the rotation device comprising:
a process chamber;
a sample holder disposed in said process chamber for holding at least one sample carrier having at least one cavity for accommodating a DNA-containing sample liquid, said sample holder having a rotation plane, and the at least one cavity having a heat input side lying in said rotation plane;
a rotational drive configured to rotate the sample holder about a rotation axis during intended operation;
a heating device configured to heat the heat input side to a high temperature value during the intended operation; and
a controller control-linked to said rotational drive and to said heating device, said controller configured to carry out the method for multiplying DNA according to claim 1.

11. The rotation device according to claim 10, wherein said heating device at least one of includes a Peltier element or is integrated into said sample holder.

12. The rotation device according to claim 10, which further comprises a cooling device configured to cool a heat output side of the cavity to a low temperature value, the heat output side disposed opposite to the heat input side.

13. The rotation device according to claim 12, wherein said cooling device includes a fan causing cooling air to flow through said process chamber.

14. A system for multiplying DNA, the system comprising:
the rotation device according to claim 10; and
the sample carrier.
